(19) United States
(12) Patent Application Publication
(10) Pub. No.: US 2026/0272456 A1
(43) Pub. Date: Sep. 17, 2026

(54) SURGICAL STAPLING APPARATUS WITH LOCKOUT ASSEMBLIES

(71) Applicant: Covidien LP, Mansfield, MA (US)

(72) Inventors: Kenneth M. CAPPOLA, Monroe, CT (US); Vincent LAFRAGOLA, JR., Killingworth, CT (US)

(21) Appl. No.: 19/168,380

(22) PCT Filed: Mar. 14, 2024
(86) PCT No.: PCT/IB2024/052469

§ 371 Date: Sep. 24, 2025

Related U.S. Application Data
(60) Provisional application No. 63/456,131, filed on Mar. 31, 2023.

(51) Int. Cl. A61B 17/072

(52) U.S. Cl. CPC A61B 17/07207; A61B 2017/07271; A61B 2017/07278

(57) ABSTRACT

A tool assembly includes an anvil assembly and a cartridge assembly pivotably supported in relation to the anvil assembly to facilitate movement of the tool assembly between an unclamped position and a clamped position. The cartridge assembly includes a staple cartridge and a channel member. The staple cartridge is received within the channel member. The staple cartridge includes a cartridge body, staples, an actuation sled, and a knife bar. The actuation sled is movable from a sled retracted position to a sled advanced position to eject the staples from the cartridge body. The channel member includes a knife guide and defines a lockout groove. The knife guide is pivotably connected to the channel member and is moveable in a transverse direction across a longitudinal axis of the tool assembly and toward the lockout groove for preventing the tool assembly from firing.

This Application claims priority from U.S. Provisional Patent Application 63/456,131, filed 31 Mar. 2023, the entire content of which is incorporated herein by reference.

### TECHNICAL FIELD

This application relates to surgical stapling apparatus and, more particularly, to surgical stapling apparatus including lockout assemblies.

### BACKGROUND

Surgical stapling apparatus configured for endoscopic use are commonly used during surgical procedures to minimize patient trauma and reduce patient recovery times. Typically, endoscopic stapling apparatus include a tool assembly and a drive assembly that is movable in relation to the tool assembly to actuate the tool assembly. The drive assembly includes a knife bar having a cutting blade for cutting tissue. The tool assembly includes anvil and cartridge assemblies that are coupled to each other by a pivot member and movable in relation to each other between unclamped and clamped positions in response to movement of the drive assembly from a retracted position to an advanced position. The cartridge assembly includes a staple cartridge that includes a cartridge body that supports staples and an actuation sled that is movable through the cartridge body in response to movement of the drive assembly from the retracted position to the advanced position to eject the staples from the cartridge body.

Some stapling apparatus include a staple cartridge that can be replaced after each firing of the stapling apparatus to facilitate reuse of the stapling apparatus. To ensure that a stapling apparatus is not fired with a spent staple cartridge, i.e., a staple cartridge having no staples, or without a staple cartridge, some stapling apparatus include lockouts.

A continuing need exists for surgical stapling apparatus having a cost-effective assembly for preventing firing of the stapling apparatus when the stapling apparatus includes a spent staple cartridge or an inoperative staple cartridge.

### SUMMARY

According to one aspect, this disclosure is directed to a surgical stapling apparatus. The surgical stapling apparatus includes a handle assembly, an adapter assembly, a drive assembly, and a tool assembly. The adapter assembly has a proximal portion and a distal portion. The proximal portion of the adapter assembly is coupled to the handle assembly. The drive assembly includes a flexible drive beam and a working member. The flexible drive beam has a distal portion secured to the working member, the working member having an I-shaped configuration and movable between a drive retracted position and a drive advanced position. The tool assembly is coupled to the distal portion of the adapter assembly and positioned to receive the working member. The tool assembly includes an anvil assembly and a cartridge assembly pivotably supported in relation to the anvil assembly to facilitate movement of the tool assembly between an unclamped position and a clamped position. The cartridge assembly includes a staple cartridge and a channel member, the staple cartridge received within the channel member. The staple cartridge includes a cartridge body, staples, an actuation sled, and a knife bar. The cartridge body defines staple receiving pockets, the staples received within the staple receiving pockets. The actuation sled is movable from a sled retracted position to a sled advanced position to eject the staples from the cartridge body. The knife bar is movable with the actuation sled from a knife retracted position to a knife advanced position. The channel member includes a flexible knife guide and defines a lockout groove. The flexible knife guide is configured to urge the working member toward the lockout groove for selectively locking out the working member.

In aspects, the knife bar may include a proximal finger that is selectively engageable with the working member to prevent the working member from engaging the lockout groove when the knife bar is disposed in a first predetermined position. When the knife bar is not disposed in the first predetermined position, or when the actuation sled is not disposed in a second predetermined position, or when the knife bar is not disposed in the first predetermined position and the actuation sled is not disposed in the second predetermined position, the flexible knife guide may be configured to urge the working member into the lockout groove. The working member may be configured to cam along the proximal finger and past the lockout groove. When the tool assembly is disposed in the clamped position and the knife bar is not disposed in the first predetermined position, the flexible knife guide may urge the working member transversely relative a longitudinal axis of the tool assembly.

In aspects, the working member may include a locking tooth that extends distally from the working member and is receivable within the lockout groove.

In aspects, the channel member may define a clearance cutout that receives a distal tab of the flexible knife guide when the working member slides along the flexible knife guide and past the locking groove.

In aspects, the anvil assembly may include an anvil and an anvil cover, the anvil cover including a second flexible knife guide that is configured to engage the working member for selectively urging the working member in a proximal relief defined in the anvil. The working member may include a first beam, a second beam, and a vertical strut that extends between the first beam and the second beam, the first beam having an angled distal edge that cooperates with a camming tip of the second flexible knife guide.

In aspects, a proximal end portion of the channel member may support a proximal lockout assembly, and the proximal lockout assembly may include a lockout bar and at least one spring supported on the lockout bar. The actuation sled may be configured to depress the lockout bar against the bias of the at least one spring to enable the working member to translate past the proximal lockout assembly.

According to another aspect, this disclosure is directed to a tool assembly for a surgical stapling apparatus. The tool assembly defines a longitudinal axis and includes an anvil assembly and a cartridge assembly. The cartridge assembly is pivotably supported in relation to the anvil assembly to facilitate movement of the tool assembly between an unclamped position and a clamped position. The cartridge assembly includes a staple cartridge and a channel member, the staple cartridge received within the channel member. The staple cartridge includes a cartridge body, staples, an actuation sled, and a knife bar. The cartridge body defines staple receiving pockets, the staples received within the staple receiving pockets. The actuation sled is movable from a sled retracted position to a sled advanced position to eject the staples from the cartridge body. The channel member includes a flexible knife guide and defines a lockout groove. The flexible knife guide is configured to urge a working member of a drive assembly in a transverse direction across the longitudinal axis of the tool assembly and toward the lockout groove for selectively locking out the working member.

In aspects, the knife bar may include a proximal finger that is configured to selectively engage the working member to prevent the working member from engaging the lockout groove when the knife bar is disposed in a first predetermined position.

In aspects, when the knife bar is not disposed in the first predetermined position, or when the actuation sled is not disposed in a second predetermined position, the flexible knife guide may be configured to urge the working member into the lockout groove. The proximal finger may be configured to enable the working member to move distally beyond the lockout groove. When the tool assembly is disposed in the clamped position and the knife bar is not disposed in the first predetermined position, the flexible knife guide may be configured to urge the working member transversely relative the longitudinal axis of the tool assembly.

In aspects, the lockout groove may be configured to receive a locking tooth of the working member to prevent distal movement of the working member through the tool assembly. The channel member may define a clearance cutout that selectively receives a distal tab of the flexible knife guide.

In aspects, the anvil assembly may include an anvil and an anvil cover, the anvil cover including a second flexible knife guide that is configured to engage the working member for selectively urging the working member in a proximal relief defined in the anvil.

In aspects, a proximal end portion of the channel member may support a proximal lockout assembly. The proximal lockout assembly may include a lockout bar and at least one spring supported on the lockout bar. The actuation sled may be configured to depress the lockout bar against the bias of the at least one spring to enable the working member to translate past the proximal lockout assembly.

According to yet another aspect, this disclosure is directed to a tool assembly. The tool assembly includes an anvil assembly and a cartridge assembly. The cartridge assembly is pivotably supported in relation to the anvil assembly to facilitate movement of the tool assembly between an unclamped position and a clamped position. The cartridge assembly includes a staple cartridge and a channel member. The staple cartridge is received within the channel member. The staple cartridge includes a cartridge body, staples, an actuation sled, and a knife bar. The actuation sled is movable from a sled retracted position to a sled advanced position to eject the staples from the cartridge body. The channel member includes a knife guide and defines a lockout groove. The knife guide is pivotably connected to the channel member. The knife guide is moveable in a transverse direction across a longitudinal axis of the tool assembly and toward the lockout groove for preventing the tool assembly from firing.

Other features of the disclosure will be appreciated from the following description.

### BRIEF DESCRIPTION OF THE DRAWINGS

The above and other aspects, features, and advantages of this disclosure will be apparent considering the following detailed description when taken in conjunction with the accompanying drawings, which are incorporated in and constitute a part of this specification, wherein:

FIG. 1 is a perspective view of a surgical stapling apparatus according to aspects of the disclosure with a tool assembly of the surgical stapling apparatus in an unclamped, pre-fired position;

FIG. 2 is a perspective view of the tool assembly of the stapling device shown in FIG. 1 with a staple cartridge of a cartridge assembly of the tool assembly separated from a channel of the cartridge assembly;

FIG. 3 is an exploded view of the staple cartridge of the cartridge assembly shown in FIG. 2;

FIG. 4 is an enlarged, perspective view of the indicated area of detail shown in FIG. 3;

FIG. 5 is an exploded view of FIG. 4;

FIG. 6 is a cross-sectional view of FIG. 4 as taken along section line 6-6;

FIG. 7 is a perspective view of the channel of the cartridge assembly of FIG. 2;

FIG. 8 is an enlarged, perspective view of the indicated area of detail shown in FIG. 7;

FIG. 9 is a perspective view, with parts separated, of an anvil assembly of the tool assembly of FIG. 1;

FIG. 10 is an enlarged top view of the indicated area of detail shown in FIG. 9;

FIG. 11 is a perspective view of the tool assembly of the surgical stapling apparatus of FIG. 1 in a clamped position with an actuation sled assembly and a drive assembly shown in broken lines for clarity;

FIG. 12 is an enlarged view of the actuation sled assembly and the drive assembly illustrated in the indicated area of detail shown in FIG. 11;

FIG. 13 is a top perspective view of a working member of the drive assembly shown in FIG. 12;

FIG. 14 is a bottom perspective view of the working member shown in FIG. 13;

FIGS. 15 and 16 are enlarged, cross-sectional views of FIG. 11 as taken along section lines 15-15 and 16-16, respectively;

FIG. 17 is a cross-sectional view of FIG. 16 as taken along section line 17-17;

FIGS. 18-20 are progressive views illustrating the working member being locked out when no knife is present;

FIGS. 21 and 22 are progressive views illustrating the working member being locked out when no sled is present;

FIG. 23 is a cross-sectional view of the tool assembly in the clamped position illustrating the working member being locked out when no sled or knife is present;

FIG. 24 is an enlarged, cross-sectional view of the tool assembly in the clamped position illustrating the working member being locked out when no cartridge is present;

FIG. 25 is an enlarged, bottom perspective view of a proximal portion of one anvil cover of the anvil assembly of FIG. 9;

FIG. 26 is a perspective view of another working member of the surgical stapling apparatus of FIG. 1;

FIGS. 27-30 are progressive views illustrating the working member of FIG. 26 cooperating with the anvil cover of FIG. 25;

FIG. 31 is a rear, perspective view of another tool assembly of the surgical stapling apparatus of FIG. 1 with a cartridge assembly of the tool assembly shown without a staple cartridge to illustrate a lockout assembly of the tool assembly;

FIG. 32 is an enlarged, perspective view of the indicated area of detail shown in FIG. 31;

FIG. 33 is an exploded view of FIG. 32; and

FIGS. 34-40 are progressive views illustrating the lockout assembly selectively effectuating lockout of the working member of FIG. 26.

### DETAILED DESCRIPTION

Aspects of this disclosure will now be described in detail with reference to the drawing figures wherein like reference numerals identify similar or identical elements. Throughout this description, the term “proximal” refers to a portion of a structure, or component thereof, that is closer to a user, and the term “distal” refers to a portion of the structure, or component thereof, that is farther from the user. Directional reference terms, such as “top,” “bottom,” “side,” “upper,” “lower,” and the like, are used to ease description of the aspects and are not intended to have any limiting effect on the ultimate orientation of a structure or any part thereof. Further, the term “clinician” is used generally to refer to medical personnel including doctors, nurses, surgeons, and support personnel.

In the following description, well-known functions or constructions are not described in detail to avoid obscuring this disclosure in unnecessary detail.

Surgical stapling apparatus of this disclosure can be utilized, for example, during laparoscopic surgical procedures that require stapling and/or transecting of tissues and/or organs of various tissue thicknesses. This disclosure relates to surgical stapling apparatus with lockout assemblies for preventing misfiring such as when no knife, no sled, and/or no cartridge is present. The disclosed surgical stapling apparatus can be configured for single or multi-use surgical stapling.

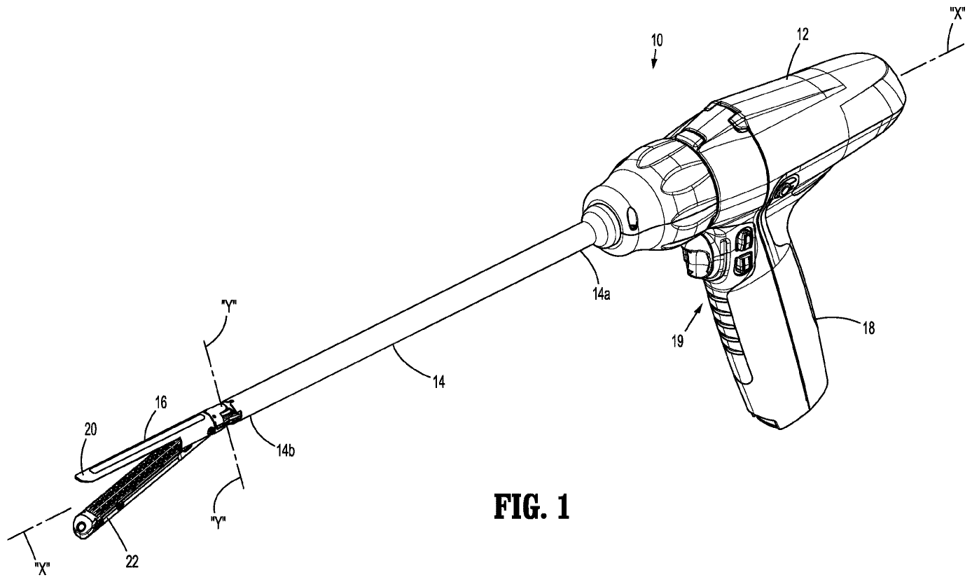

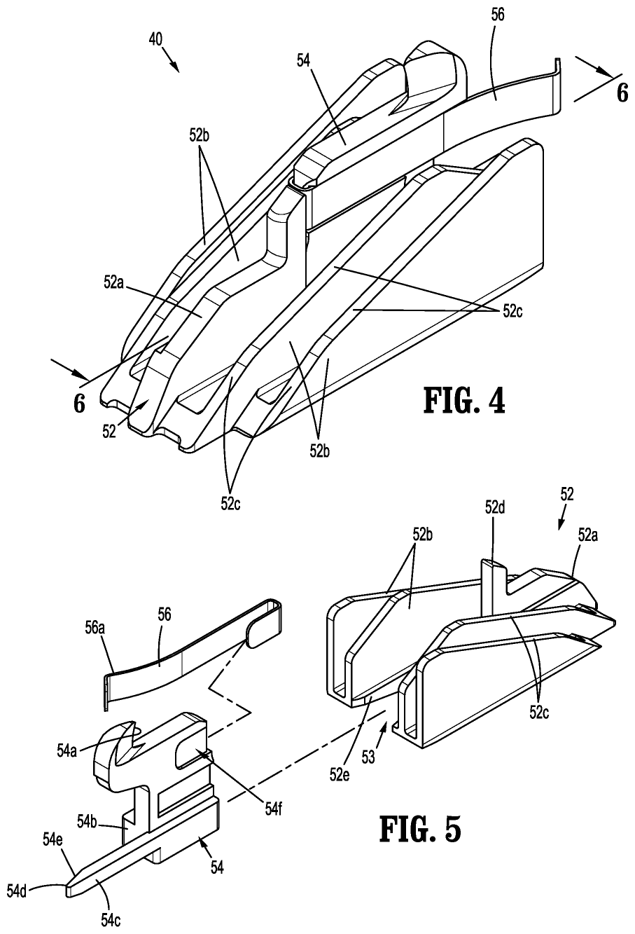

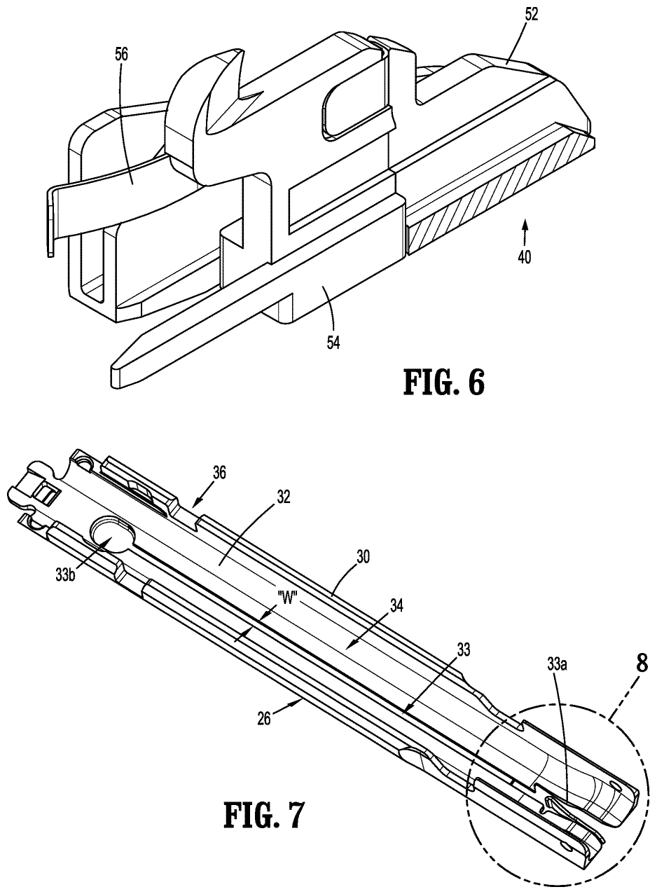

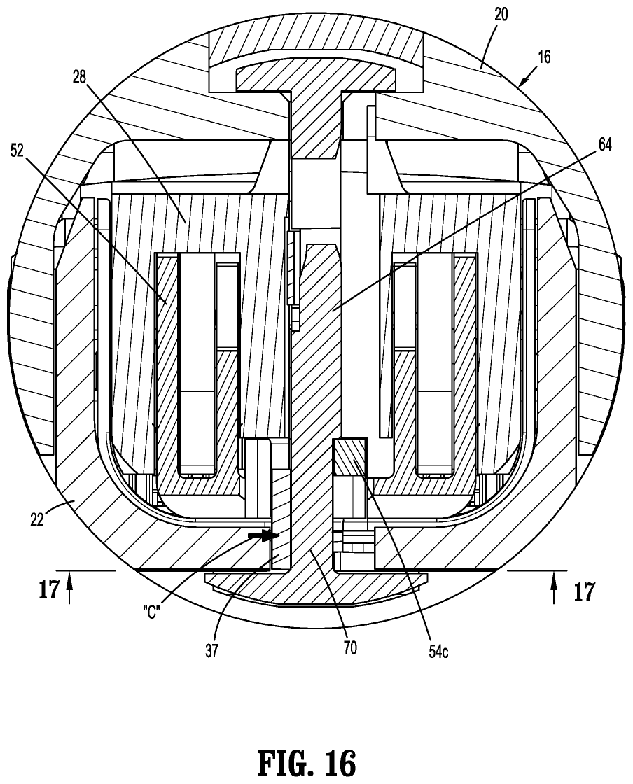

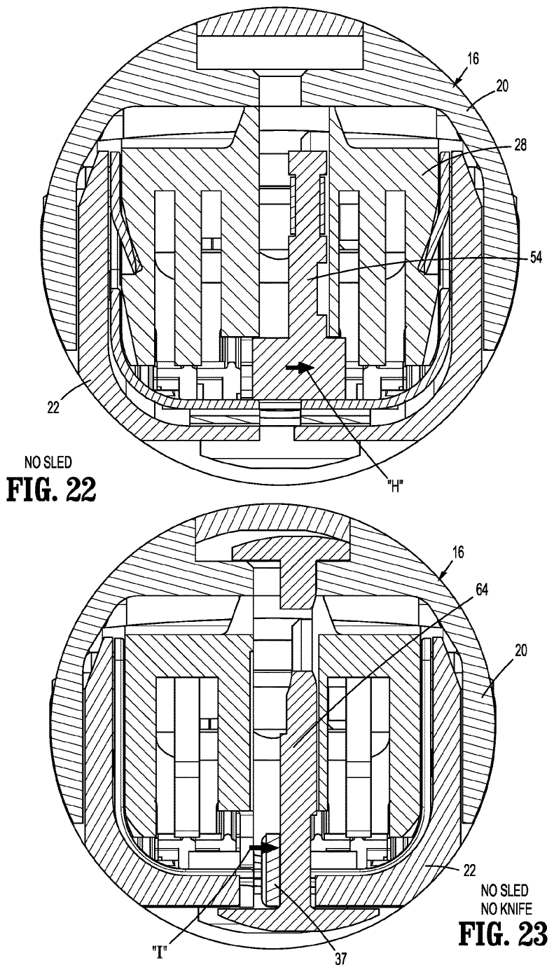

With reference to FIGS. 1-24, a surgical stapling apparatus, shown generally as stapling apparatus 10 that includes a handle assembly 12, an adapter assembly 14, and a tool assembly 16.

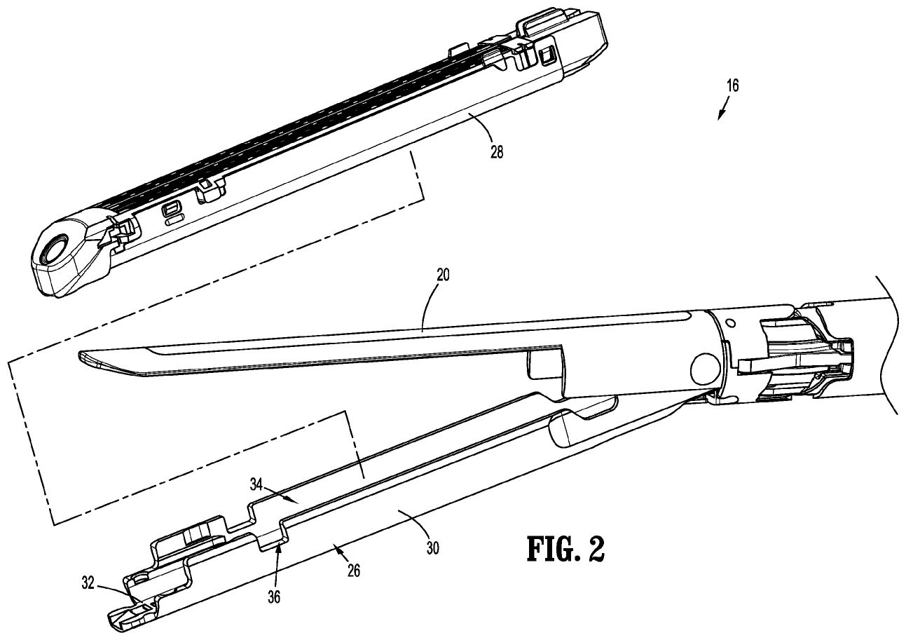

As best seen in FIGS. 1-2, the handle assembly 12 is powered and includes a stationary handgrip 18 and an actuation button or buttons 19. The actuation buttons 19 are operable to actuate various functions of the tool assembly 16 via the adapter assembly 14, e.g., approximation of the tool assembly 16, and stapling and cutting of tissue. In certain aspects of the disclosure, the handle assembly 12 supports batteries (not shown) that provide power to the handle assembly 12 to operate the stapling apparatus 10. Although the stapling device 10 is illustrated as a powered stapling device, it is envisioned that the advantages of this disclosure are suitable for use with manually powered surgical stapling apparatus as well as robotically controlled stapling apparatus.

The adapter assembly 14 of surgical stapling apparatus 10 defines a longitudinal axis “X” and includes a proximal portion 14a and a distal portion 14b. The proximal portion 14a of the adapter assembly 14 is coupled to the handle assembly 12, and the distal portion 14b of the adapter assembly 14 supports the tool assembly 16. In aspects of the disclosure, the tool assembly 16 is pivotably coupled to the distal portion 14b of the adapter assembly 14 to facilitate articulation of the tool assembly 16 in relation to the adapter assembly 14 about an articulation axis “Y”.

The tool assembly 16 of surgical stapling apparatus 10 includes an anvil assembly 20 and a cartridge assembly 22. In aspects of the disclosure, the cartridge assembly 22 is pivotably supported in relation to the anvil assembly 20 to facilitate movement of the tool assembly 16 between an unclamped position (FIG. 1) and a clamped position (FIG. 11). Alternately, the anvil assembly 20 can be pivotable towards the cartridge assembly 22 to move the tool assembly 16 relative to the clamped position.

As seen in FIGS. 2, 3, and 7, the cartridge assembly 22 of tool assembly 16 includes a channel member 26 and a staple cartridge 28. The channel member 26 includes side walls 30 and a bottom wall 32 that define a cavity 34. The bottom wall 32 defines an elongated slot 33 through the channel member 26. The channel member 26 includes a guide portion 33a defined through the bottom wall 32 on a proximal end of the elongated slot 33. The guide portion 33a includes a first inner wall 33b and a second inner wall 33c that spaced-apart from and facing the first inner wall 33b of the guide portion 33a. The elongated slot 33 extends distally to a distal opening 33b defined through the bottom wall 32 of the channel member 26 at a distal end of the elongated slot 33. The distal opening 33b is wider than a width “W” of the elongated slot 33. The staple cartridge 28 is removably received within the cavity 34 of the channel member 26 and is replaceable to facilitate reuse of the stapling apparatus 10. Each of the side walls 30 of the channel member 26 has an upper end nearest the anvil assembly 20 that defines a recess 36.

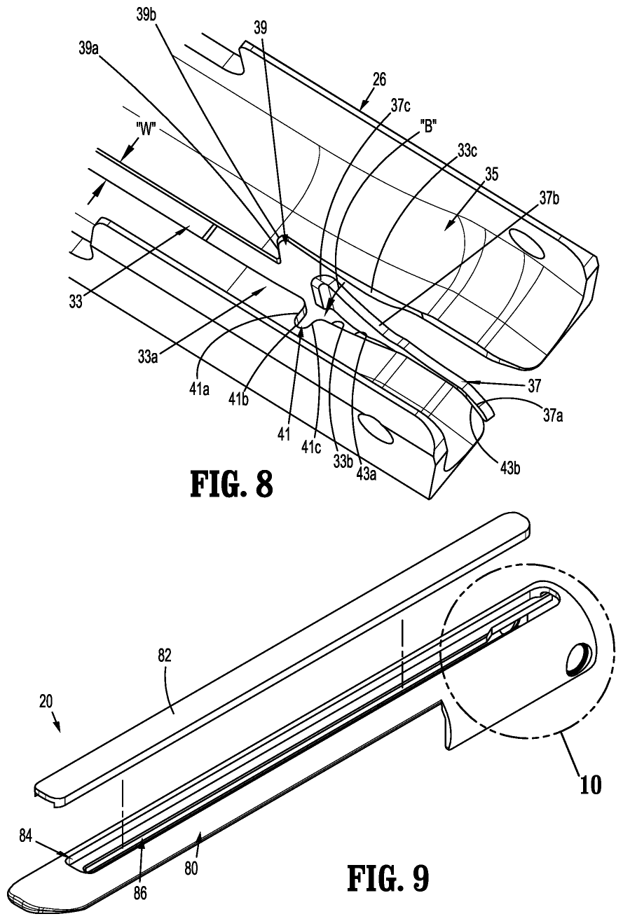

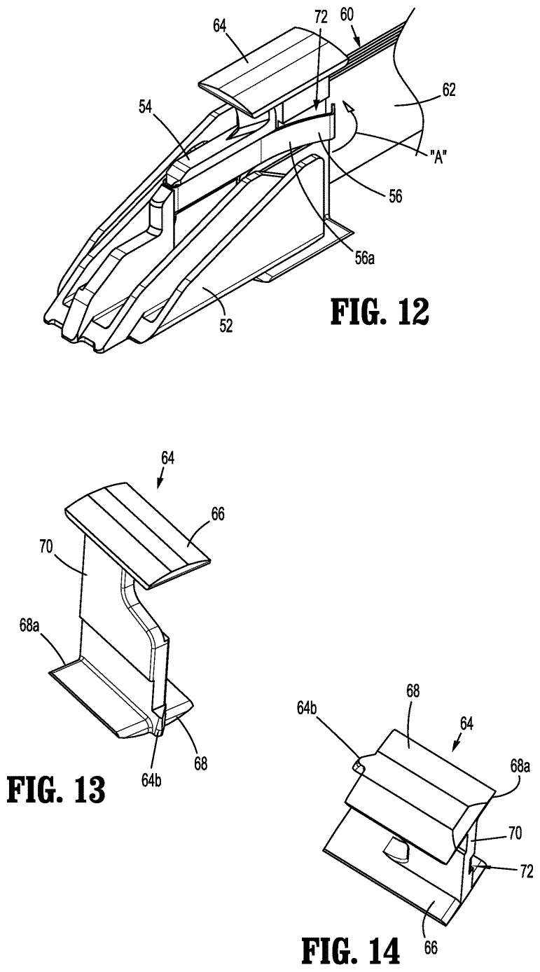

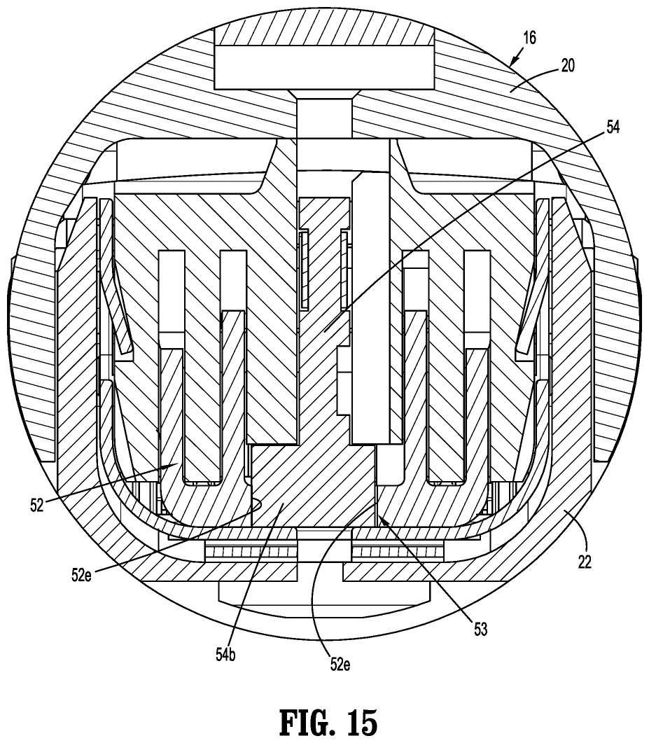

Referring briefly to FIG. 8, the channel member 26 of the cartridge assembly 22 further includes a lockout assembly 35 that is configured to cooperate with a drive assembly 60 of the stapling apparatus 10 (see FIGS. 12 and 17-19) to prevent misfiring when no knife bar 54, no actuation sled 52, and/or no staple cartridge 28 is present (see FIGS. 15-24). The lockout assembly 35 includes a flexible knife guide 37 secured to the first inner wall 33b of the guide portion 33a, a lockout groove 39 defined in the second inner wall 33c of the guide portion 33a, and a clearance cutout 41 defined in the first inner wall 33b. The lockout groove 39 is distal to the clearance cutout 41 and is laterally recessed into the second inner wall 33c of the bottom wall 32 from the elongated slot 33. The lockout groove 39 includes an angled edge 39a and a bend 39b that provide a transition surface along the second inner wall 33c between the elongated slot 33 and the guide portion 33a. Similarly, the clearance cutout 41 is laterally recessed into the first inner wall 33b of the bottom wall 32 from the elongated slot 33. The clearance cutout 41 includes a long segment 41a, a radius 41b extending from the long segment 41a, and a short segment 41c extending from the radius 41b. The clearance cutout 41 provides a transition surface along the first inner wall 33b between the elongated slot 33 and the guide portion 33a. The first inner wall 33b further includes a proximal surface 43a that connects to the short segment 41c of the clearance cutout 41 and a mounting surface 43b that is proximal to the proximal surface 43a. The flexible knife guide 37 includes a proximal mounting portion 37a that is secured to the mounting surface 43b of the first inner wall 33b, and a flexible arm 37b that extends distally from the proximal mounting portion 37a. In aspects, the proximal mounting portion 37a of the flexible knife guide 37 may be welded to the mounting surface 43b of the first inner wall 33b. The flexible arm 37b is cantilevered from the first inner wall 33b and moveable relative to the proximal mounting portion 37a between a flexed position and an unflexed position, as indicated by arrow “B”. The flexible arm 37b extends distally to a distal tab 37c that aligns with and is selectively receivable within the clearance cutout 41 when the flexible arm is moved from the unflexed position to the flexed position.

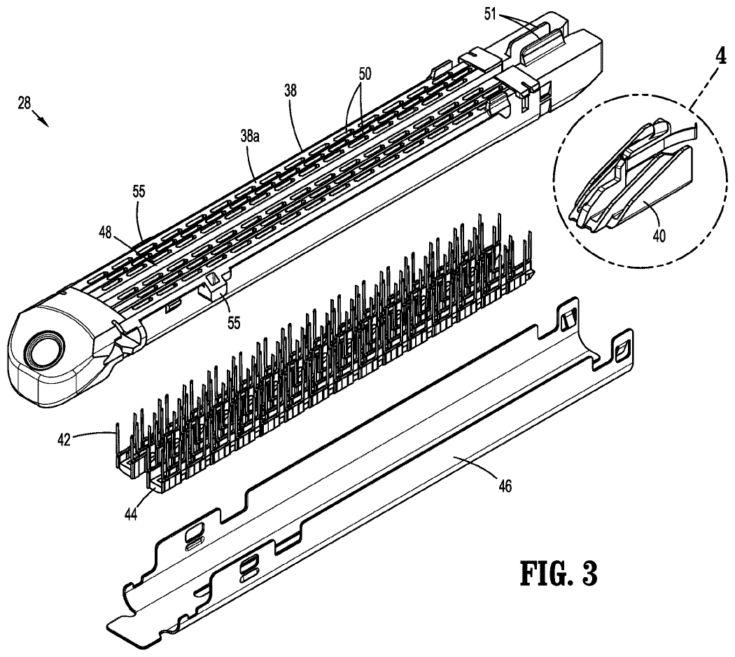

With continued reference to FIGS. 2 and 3, the staple cartridge 28 of tool assembly 16 includes a cartridge body 38, an actuation sled assembly 40, staples 42, pushers 44, and a staple guard 46. The cartridge body 38 defines a knife slot 48 and staple receiving pockets 50 that are positioned on each side of the knife slot 48. The cartridge body 38 includes knife guards 51 that are positioned on opposite sides of the knife slot 48 at a position proximal of the staple receiving pockets 50. In aspects of the disclosure, the staple receiving pockets 50 are aligned in two or more rows on the opposite sides of the knife slot 48. In some aspects of the disclosure, the knife slot 48 is centrally located in the cartridge body 38, although it is envisioned that the knife slot 48 could be offset from the central axis of the cartridge body 38.

The cartridge body 38 of staple cartridge 28 includes a tissue engaging surface 38a and laterally extending protrusions 55 that are received within the recesses 36 (FIG. 2) of the channel member 26 to properly position the staple cartridge 28 within the cavity 34 of the channel member 26. The staples 42 and the pushers 44 are received within the staple receiving pockets 50 of the cartridge body 38, and the staple guard 46 is secured to the bottom of the cartridge body 38 to retain the staples 42 and pushers 44 within the cartridge body 38.

With reference to FIGS. 3-6, the actuation sled assembly 40 of staple cartridge 28 includes an actuation sled 52, a knife bar 54 received within the actuation sled 52, and a latch 56 coupled to the knife bar 54 for selectively coupling the knife bar 54 to a distal portion of the drive assembly 60 (see FIG. 12). The latch 56, which may be in the form of a leaf spring, may be formed of a resilient material such as spring steel. The actuation sled 52 includes a central body portion 52a and cam members 52b that are positioned on opposite sides of the central portion 52a. The actuation sled 52 further includes inner surfaces 52e that define a knife bar cavity 53 for receiving the knife bar 54 and for restricting lateral movement of the knife bar 54. The central portion 52a is received within the knife slot 48 (FIG. 3) of the cartridge body 38. The cam members 52b define ramps 52c that are positioned to engage the pushers 44 supported within the cartridge body 38 as the actuation sled 52 moves from a sled retracted position towards a sled advance position to sequentially eject the staples 42 from the cartridge body 38. When the actuation sled 52 is in the sled retracted position, the knife bar 54 is positioned between the knife guards 51 of the cartridge body 38. The actuation sled 52 further includes a post 52d that abuts the knife bar 54 and supports the latch 56 between the actuation sled 52 and the knife bar 54.

The knife bar 54 of the actuation sled assembly 40 is positioned proximal to the actuation sled 52 and is movable within the knife slot 48 of the cartridge body 38 between a knife retracted position and a knife advanced position. The knife bar 54 includes a cutting edge 54a that is positioned in a gap defined between the tissue engaging surface 38a of the cartridge body 38 and the anvil assembly 20 when the tool assembly 16 (FIG. 1) is in the clamped position to cut tissue clamped between the staple cartridge 28 and the anvil assembly 20. The knife bar 54 also includes a base 54b that is positioned on a lower end of the knife bar 54 opposite to the cutting edge 54a. The base 54b of the knife bar 54 is positioned to move along an inner surface of a bottom wall of the staple guard 46 when the knife bar 54 is moved from the knife retracted position to the knife advanced position. The actuation sled 52 is positioned distally of the knife bar 54 and is engaged by the knife bar 54 when the knife bar 54 is moved from the knife retracted position to the knife advanced position within the cartridge body 38 to move the actuation sled 52 from the sled retracted position to the sled advanced position within the cartridge body 38 into sequential engagement with the pushers 44 to urge the pushers 44 upwardly and eject the staples 42 from the staple receiving pockets 50 of the cartridge body 38. The knife bar 54 further includes a proximal finger 54c that extends proximally from the base 54b and extends proximally to a camming tip 54d having an angled cam edge 54e that faces inwardly toward a center of the knife bar 54. The knife bar 54 also defines a latch slot 54f that receives a distal portion of the latch 56.

With reference to FIGS. 12-14, a distal portion of the drive assembly 60 of the stapling device 10 (FIG. 1) includes a flexible drive beam 62 and a working member 64. The flexible drive beam 62 has a distal portion that is secured to a proximal portion of the working member 64. The working member 64 may be in the form of an I-beam having an I-shaped configuration. The working member 64 includes a first beam 66, a second beam 68, and a vertical strut 70 that extends between the first and second beams 66, 68. The vertical strut 70 defines a latch channel 72 that is positioned to receive a deformable hook member 56a of latch 56 to selectively couple the knife bar 54 to the working member 64 of the drive assembly 60 when the hook member 56a moves from a non-deformed state to a deformed state, as indicated by arrow “A” (FIG. 12).

In aspects of the disclosure, the hook member 56a of the latch 56 moves from the non-deformed state to the deformed state when the actuation sled assembly 40 moves through the cartridge assembly 22. For instance, as the knife bar 54 moves from the knife retracted position towards the knife advanced position, the latch 56 moves into the knife slot 48 of the cartridge body 38 to move the latch 56 from the non-deformed state to the deformed state. In the deformed state, the knife bar 54 is coupled to the working member 64 such that movement of the drive assembly 60 between the drive retracted position and the drive advanced position moves the knife bar 54 between the knife retracted position and the knife advanced position.

The first beam 66 of the working member 64 is slidably received within the anvil assembly 20 for enabling the first beam 66 to slide along the anvil assembly 20, and the second beam 68 of the working member 64 is slidably received within the cartridge assembly 22 for enabling the second beam 68 to slide along the cartridge assembly 22. The second beam 68 includes a proximal cam surface 68a on a proximal end portion of the second beam 68. The working member 64 further includes locking tooth 64b that extends distally from the working member 64 on a lower end portion of the working member 64. The locking tooth 64b is angled in a lateral direction relative to the vertical strut 70a.

The drive assembly 60 of the stapling device 10 is movable between a drive retracted position and a drive advanced position via a drive clamped position. When the drive assembly 60 is moved from the drive retracted position to the drive clamped position, the first beam 66 engages the anvil assembly 20 and the second beam 68 engages the cartridge assembly 22 to move the tool assembly 16 (FIG. 1) from the unclamped position to the clamped position. When the drive assembly 60 moves from the drive clamped position to the drive advanced position, a distal portion of the vertical strut 70 of the working member 64 of the drive assembly 60 abuts the knife bar 54 to advance the knife bar 54 into engagement with the actuation sled 52 and advance the knife bar 54 and the actuation sled 52 from their retracted positions to their advanced positions to eject the staples 42 from the cartridge body 38 and cut tissue clamped between the anvil assembly 20 and the cartridge assembly 22. When the stapling device 10 is fired and the drive assembly 60 is moved from the drive advanced position to the drive retracted position, the actuation sled 52 remains in the sled advanced position.

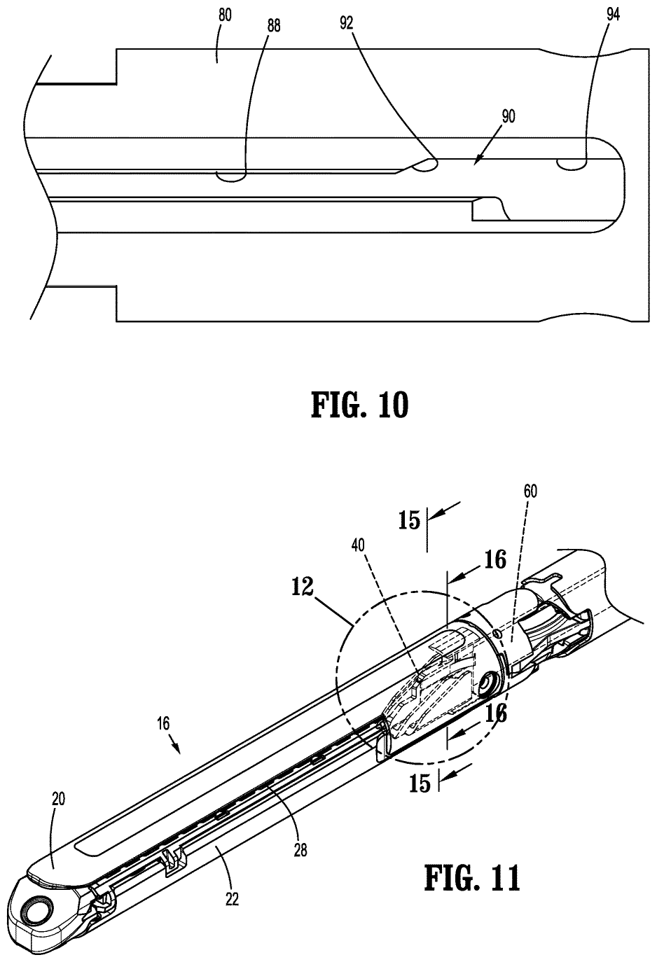

With reference to FIGS. 9 and 10, the anvil assembly 20 includes an anvil 80 and an anvil cover 82 that is received within a cover cavity 84 defined in an upper surface of the anvil 80. The anvil 80 further includes an elongated channel 86 that extends along the cover cavity 84 and is defined through the anvil 80 by an inner surface 88. The elongated channel 86 includes a proximal relief 90 defined by an outwardly angled surface 92 and a proximal linear surface 94. The proximal relief 90 is configured to enable transverse movement of drive assembly 60.

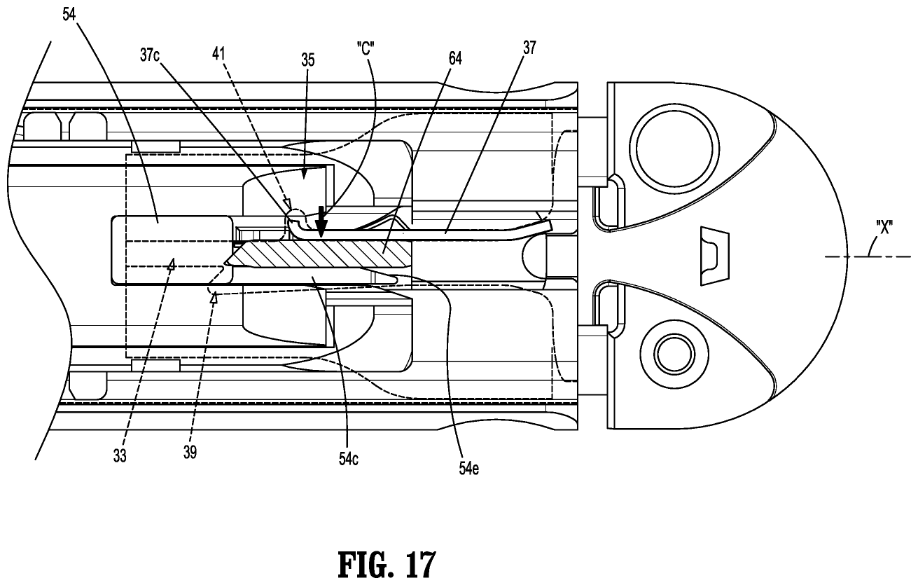

With reference to FIGS. 15-24, the lockout assembly 35 prevents misfiring when no knife bar 54, no actuation sled 52, and/or no staple cartridge 28 is present. As best seen in FIGS. 15-17, when the tool assembly 16 is disposed in the clamped position and the surgical stapling apparatus 10 is completely and properly assembled, transverse movement of the knife bar 54 is restricted by the inner surfaces 52e of the actuation sled 52 defining the knife bar cavity 53, and the proximal finger 54c of the knife bar 54 prevents the working member 64 of the drive assembly 60 from moving transversely relative to the longitudinal axis “X” against the transverse biasing forces applied by the flexible knife guide 37 against the sidewall of the vertical strut 70, as indicated by arrows “C”. In particular, as seen in FIG. 17, the working member 64 of the drive assembly 60 cams along the angled cam edge 54e of the proximal finger 54c of the knife bar 54 and slides along the side surfaces of the proximal finger 54c and past the lockout groove 39 defined in the channel member 26 of the cartridge assembly 22 such that the working member 64 remains aligned with, and movable through, the elongated slot 33 defined through the bottom wall 32 of the cartridge assembly 22 when driven distally by the flexible drive beam 62 of drive assembly 60.

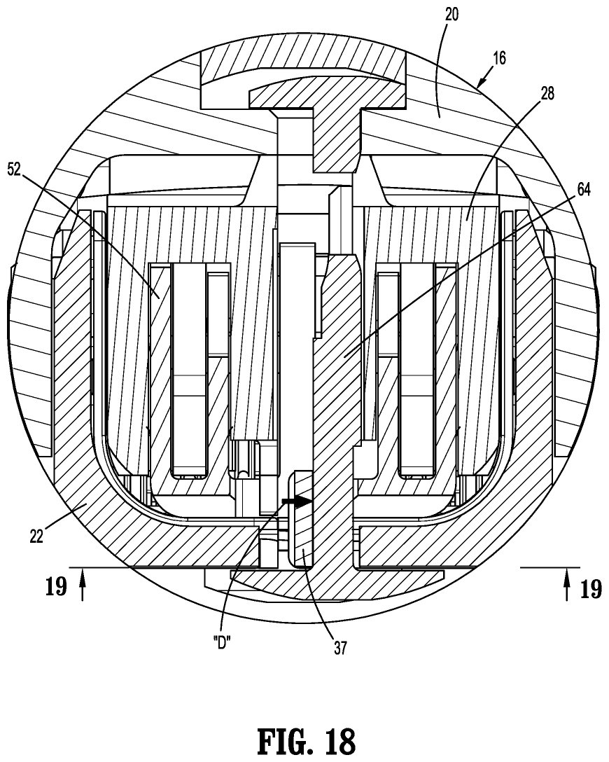

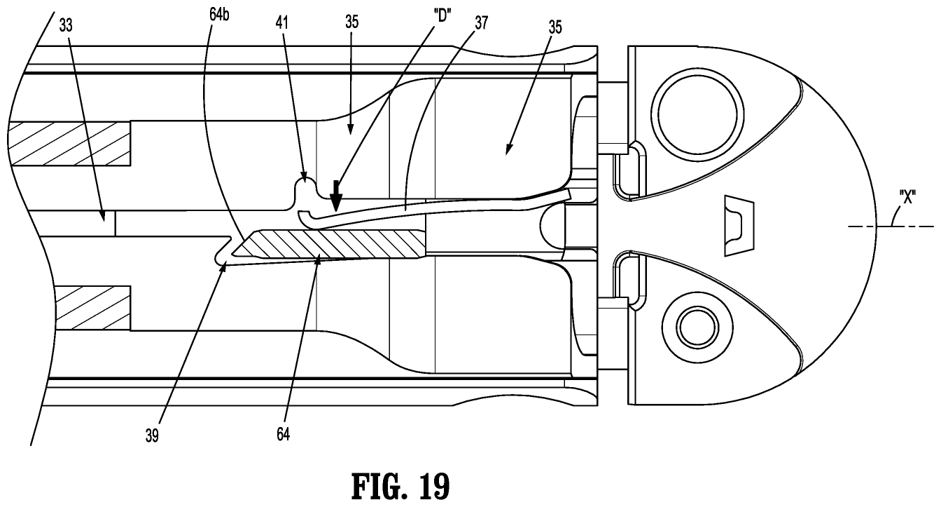

With reference to FIGS. 18 and 19, when the tool assembly 16 is disposed in the clamped position and no knife bar 54 is present, the flexible knife guide 37 urges the working member 64 of the drive assembly 60 transversely relative to the longitudinal axis “X”, as indicated by arrow “D”. As the working member 64 continues to move distally and transversely across the tool assembly 16, as indicated by arrow “E”, under the biasing forces of the flexible knife guide 37, the working member 64, namely, the locking tooth 64b of the working member 64 is driven into the lockout groove 39 of the lockout assembly 35 to prevent further distal movement of the working member 64 and/or to lock out the working member 64. The resulting angled contact between the locking tooth 64b and the locking groove 39 enables increased locking engagement under load.

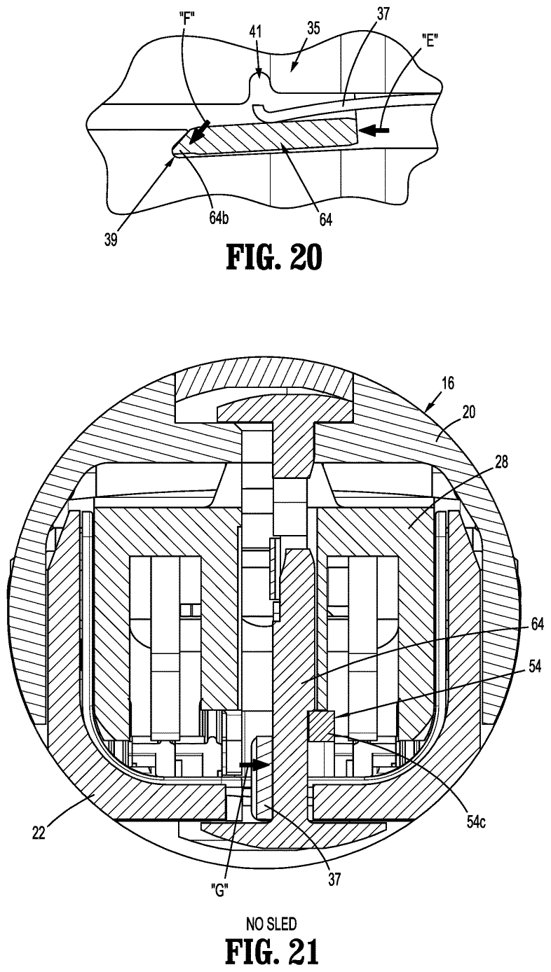

As seen in FIGS. 21-23, when the tool assembly 16 is disposed in the clamped position and no actuation sled 52 and/or no knife bar 54 is/are present, the flexible knife guide 37 urges the working member 64 of the drive assembly 60 and/or the knife bar 54 transversely relative to the longitudinal axis “X”, as indicated by arrows “G”, “H”, and/or “I”, respectively, so that the locking tooth 64b of the working member 64 is driven into the lockout groove 39 of the lockout assembly 35 as shown in FIG. 20 and detailed above.

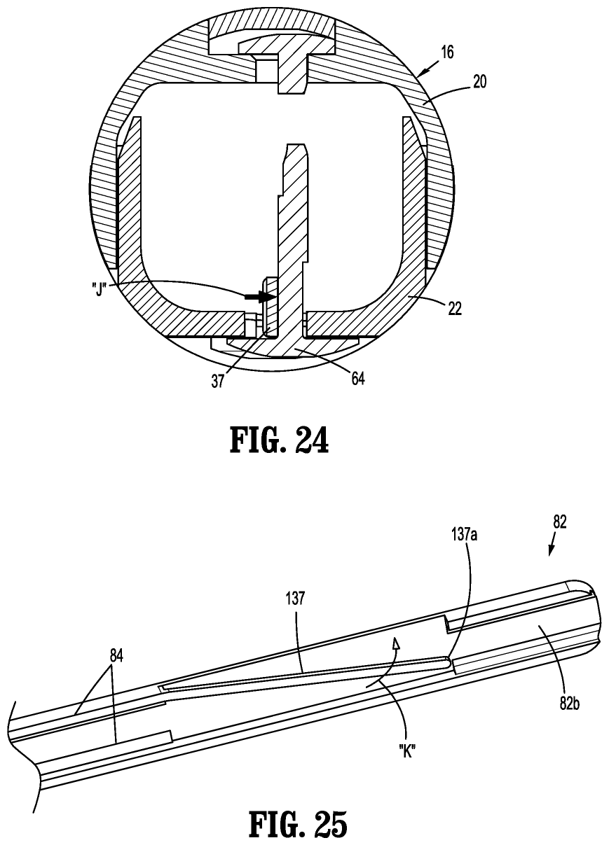

Similarly, FIG. 24 illustrates when the tool assembly 16 is disposed in the clamped position and no staple cartridge 28 is present. Specifically, the flexible knife guide 37 urges the working member 64 of the drive assembly 60 transversely relative to the longitudinal axis “X”, as indicated by arrows “J”, so that the locking tooth 64b of the working member 64 is driven into the lockout groove 39 of the lockout assembly 35 as shown in FIG. 20 and detailed above.

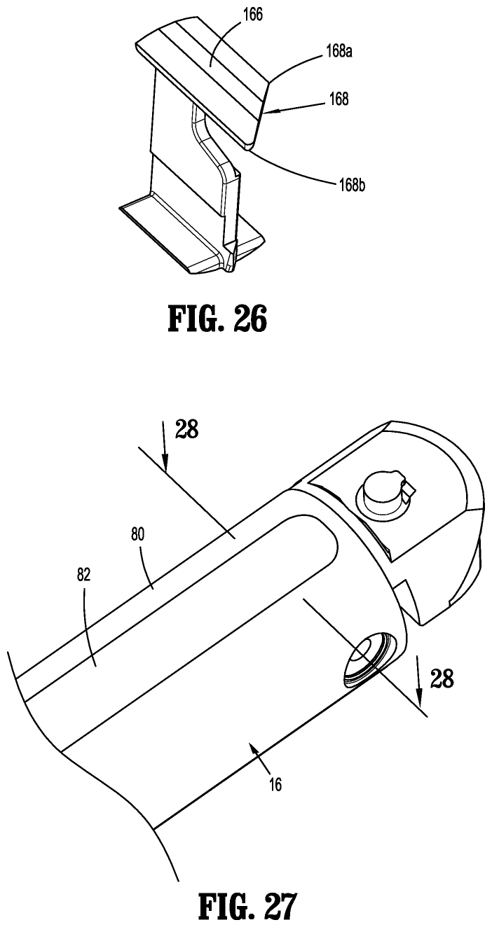

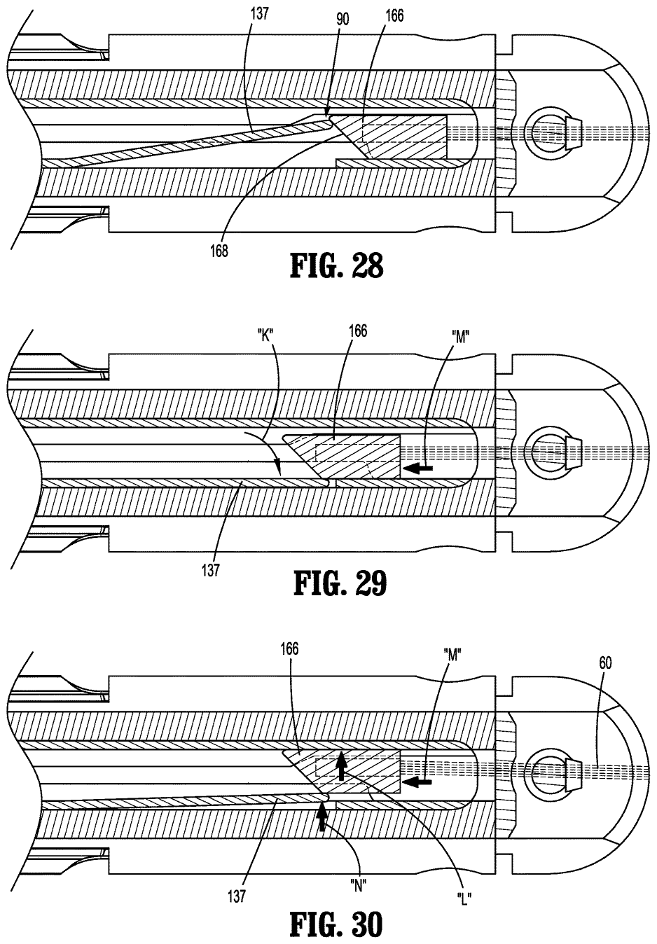

Turning now to FIGS. 25-30, the lockout assembly 35 can additionally and/or alternatively include a flexible knife guide 137 that is flexibly coupled to a rail 84 disposed on a bottom surface 82b of the anvil cover 82, as indicated by arrows “K”. The flexible knife guide 137 extends to a camming tip 137a. In aspects, working member 64 can further include a first beam 166 having an angled distal edge 168 that extends between a proximal corner 168a and a distal corner 168b. The angled distal edge 168 is configured to cooperate with the camming tip 137a of the flexible knife guide 137 of the anvil cover 82 to urge the working member 64 toward the proximal relief 90, as indicated by arrow “L”, so that that the locking tooth 64b of the working member 64 is driven into the lockout groove 39 of the lockout assembly 35 when no knife bar 54, no actuation sled 52, and/or no staple cartridge 28 is present, as shown in FIG. 20, to lock out the working member 64. Indeed, the camming tip 137a of the flexible knife guide 137 cams along angle distal edge 168 in response to distal driving force “M” of the drive assembly 60 until the camming tip 137a cams over the proximal corner 168a of the angled distal edge 168. When no knife bar 54, no actuation sled 52, and/or no staple cartridge 28 is present, the flexible knife guide 137 imparts a biasing force “N” that urges the flexible knife guide 137 toward the proximal relief 90 (see FIG. 10). When the knife bar 54, the actuation sled 52, and the staple cartridge 28 are present, the drive assembly 60 urges the working member 64 distally past the lockout assembly 35 and the proximal relief 90 so that the surgical stapling apparatus 10 can be fired.

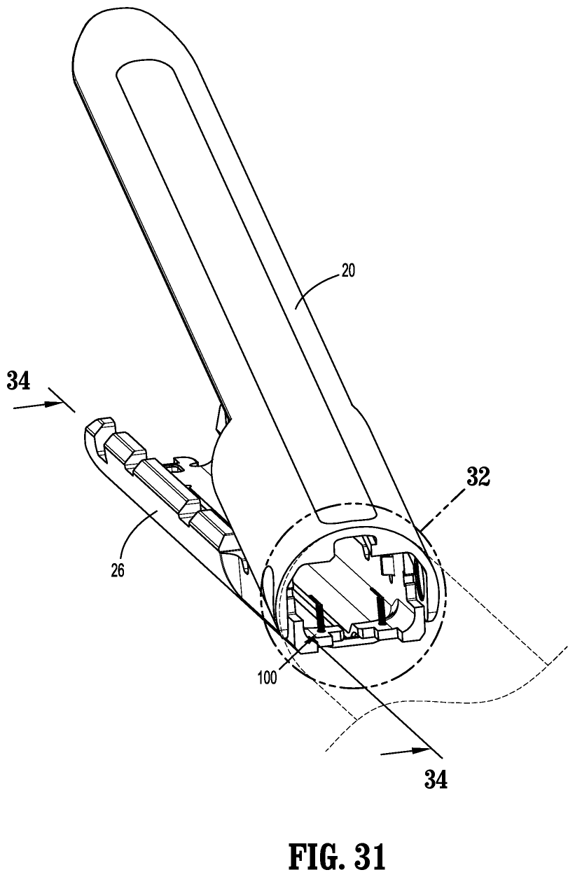

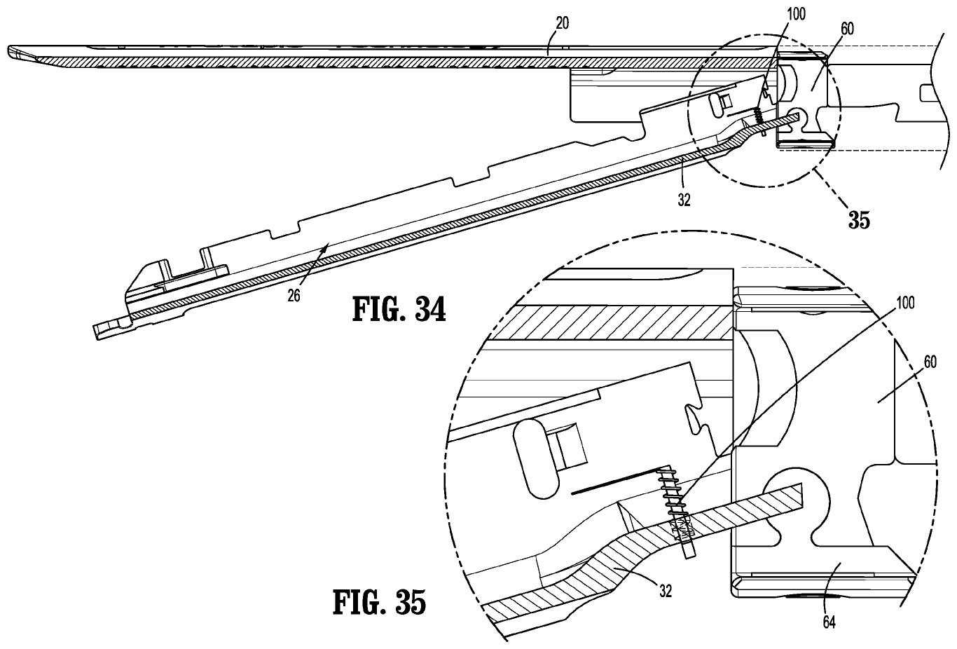

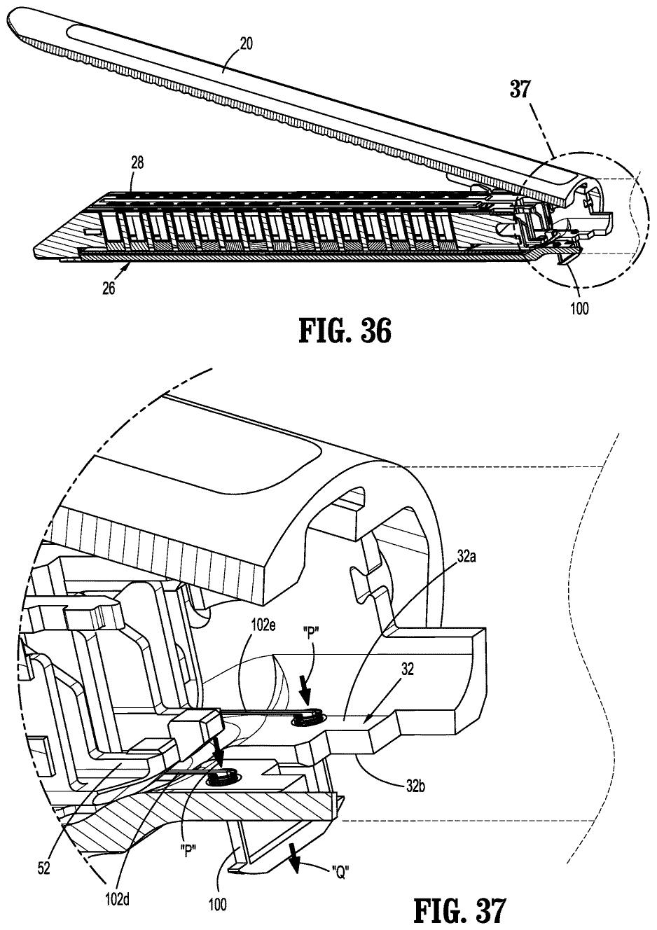

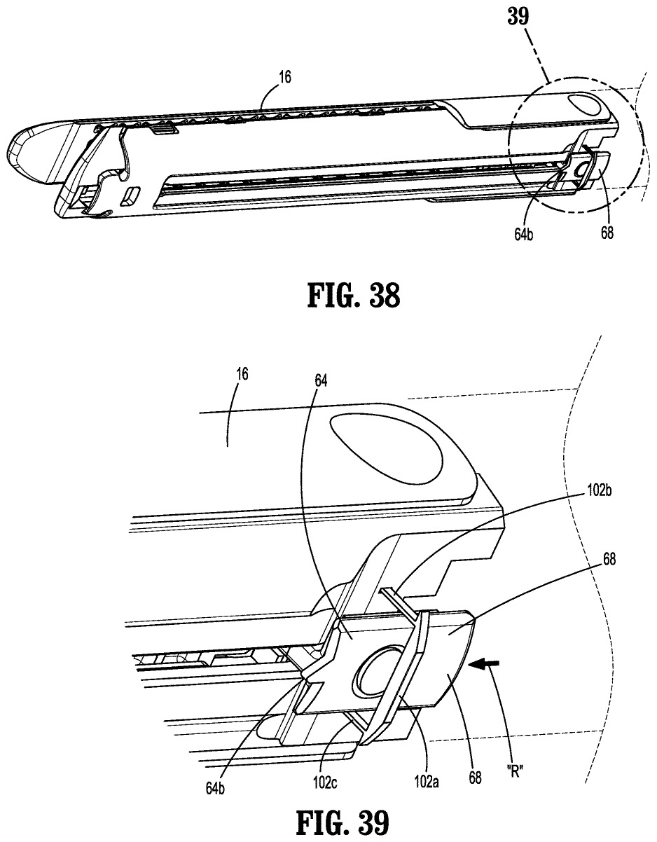

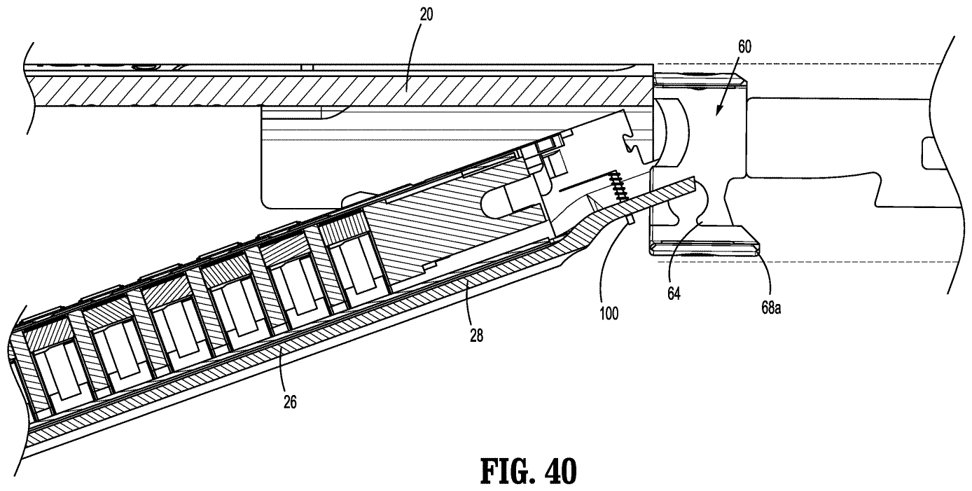

With reference to FIGS. 31-40, additionally and/or alternatively, the lockout assembly 35 can include a proximal lockout assembly 100 coupled to a proximal end portion of the bottom wall 32 of the channel member 26. The proximal lockout assembly 100 includes a lockout bar 102 and springs 104 supported on the lockout bar 102. The lockout bar 102 includes a transverse bar 102a disposed below the bottom wall 32, a first leg 102b, and a second leg 102c that extend vertically upward from the transverse bar 102a. The first leg 102b includes a first engagement arm 102d that extends distally from an upper end portion of the first leg 102b, and the second leg 102c includes a second engagement arm 102e that extends distally from an upper end portion of the second leg 102c. Each of the first and second legs 102b, 102c is received within a respective one of the springs 104. The bottom wall 32 defines holes 106 therethrough that receive the first and second legs 102b, 102, and springs 104 therein. As seen in FIG. 37, when the staple cartridge 28 is received within the channel member 26, the proximal end portion of the actuation sled 52 depresses the first and second engagement arms 102d, 102e downwardly toward the bottom wall 32 and against the biasing forces of the springs 104, as indicated by arrows “P”, so that the first and second legs 102b, 102c space the transverse bar 102a from the bottom surface 32b of the bottom wall 32, as indicated by arrow “Q”. In such position, the second beam 68 of the working member 64 can pass over the transverse bar 102a and beneath the bottom surface 32b of the bottom wall 32 to enable the working member 64 to distally advance through the tool assembly 16, as indicated by arrow “R”.

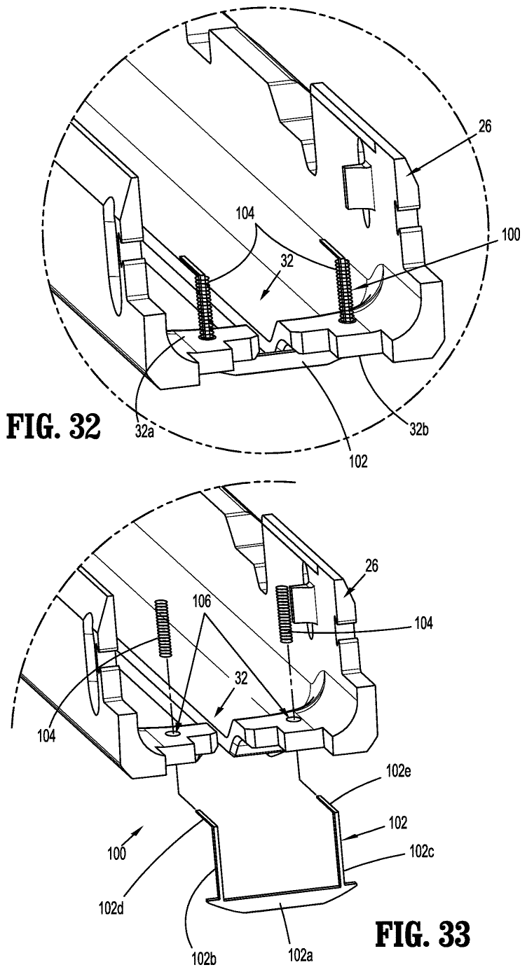

If the staple cartridge 28 is not received within the channel member 26, the springs 104 maintain the first and second engagement arms 102d, 102e in a raised position away from the top surface 32a of the bottom wall 32 (see FIG. 32) so that the transverse bar 102a prevents the second beam 68 of the working member 64 from moving past the cartridge lockout assembly 100. Then, once the actuation sled 52 is moved distally away from the proximal lockout assembly 100, the springs 104 urge the proximal lockout assembly 100 back to its initial position where the transverse bar 102a is disposed adjacent to (e.g., in contact with) the bottom surface 32b of the bottom wall 32 of the channel member 26. The proximal cam surface 68a of the working member 64 (or other camming feature supported on a proximal surface of working member 64) are configured to engage the proximal lockout assembly 100 to urge the proximal lockout assembly 100 downwardly as the working member 64 is retracted to enable the working member 64 to return to its initial proximal position. Once proximally past the proximal lockout assembly 100, the springs 104 again urge the proximal lockout assembly 100 back to its initial position.

As can be appreciated, securement of any of the components of the presently disclosed apparatus can be effectuated using known securement techniques such welding, crimping, gluing, fastening, etc.

The various aspects disclosed herein may also be configured to work with robotic surgical systems and what is commonly referred to as “Telesurgery.” Such systems employ various robotic elements to assist the clinician and allow remote operation (or partial remote operation) of surgical instrumentation. Various robotic arms, gears, cams, pulleys, electric and mechanical motors, etc. may be employed for this purpose and may be designed with a robotic surgical system to assist the clinician during an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients. For a detailed description of exemplary medical workstations and/or components thereof, reference may be made to U.S. Pat. No. 8,828,023, and PCT Application Publication No. WO2016/025132, the entire contents of each of which are incorporated by reference herein.

Persons skilled in the art will understand that the structures and methods specifically described herein and illustrated in the accompanying figures are non-limiting exemplary aspects, and that the description, disclosure, and figures should be construed merely as exemplary of aspects. It is to be understood, therefore, that the present disclosure is not limited to the precise aspects described, and that various other changes and modifications may be effected by one skilled in the art without departing from the scope or spirit of the disclosure. Additionally, it is envisioned that the elements and features illustrated or described in connection with one exemplary aspect may be combined with the elements and features of another without departing from the scope of the present disclosure, and that such modifications and variations are also intended to be included within the scope of the present disclosure. Indeed, any combination of any of the presently disclosed elements and features is within the scope of the present disclosure. Accordingly, the subject matter of the present disclosure is not to be limited by what has been particularly shown and described.

What is claimed is:

1. A surgical stapling apparatus, comprising:
a handle assembly;
an adapter assembly having a proximal portion and a distal portion, the proximal portion of the adapter assembly coupled to the handle assembly; and
a drive assembly including a flexible drive beam and a working member, the flexible drive beam having a distal portion secured to the working member, the working member having an I-shaped configuration and movable between a drive retracted position and a drive advanced position; and
a tool assembly coupled to the distal portion of the adapter assembly and positioned to receive the working member, the tool assembly including:
an anvil assembly; and
a cartridge assembly pivotably supported in relation to the anvil assembly to facilitate movement of the tool assembly between an unclamped position and a clamped position, the cartridge assembly including a staple cartridge and a channel member, the staple cartridge received within the channel member, the staple cartridge including a cartridge body, staples, an actuation sled, and a knife bar, the cartridge body defining staple receiving pockets, the staples received within the staple receiving pockets, the actuation sled movable from a sled retracted position to a sled advanced position to eject the staples from the cartridge body, the knife bar movable with the actuation sled from a knife retracted position to a knife advanced position, the channel member including a flexible knife guide and defining a lockout groove, the flexible knife guide configured to urge the working member toward the lockout groove for selectively locking out the working member.

2. The surgical stapling apparatus of claim 1, wherein the knife bar includes a proximal finger that is selectively engageable with the working member to prevent the working member from engaging the lockout groove when the knife bar is disposed in a first predetermined position.

3. The surgical stapling apparatus of claim 2, wherein when the knife bar is not disposed in the first predetermined position, or when the actuation sled is not disposed in a second predetermined position, the flexible knife guide is configured to urge the working member into the lockout groove.

4. The surgical stapling apparatus of claim 3, wherein the working member is configured to cam along the proximal finger and past the lockout groove.

5. The surgical stapling apparatus of claim 3, wherein when the tool assembly is disposed in the clamped position and the knife bar is not disposed in the first predetermined position, the flexible knife guide urges the working member transversely relative a longitudinal axis of the tool assembly.

6. The surgical stapling apparatus of claim 1, wherein the working member includes a locking tooth that extends distally from the working member and is receivable within the lockout groove.

7. The surgical stapling apparatus of claim 4, wherein the channel member defines a clearance cutout that receives a distal tab of the flexible knife guide when the working member slides along the flexible knife guide and past the locking groove.

8. The surgical stapling apparatus of claim 1, wherein the anvil assembly includes an anvil and an anvil cover, the anvil cover including a second flexible knife guide that is configured to engage the working member for selectively urging the working member in a proximal relief defined in the anvil.

9. The surgical stapling apparatus of claim 8, wherein the working member includes a first beam, a second beam, and a vertical strut that extends between the first beam and the second beam, the first beam having an angled distal edge that cooperates with a camming tip of the second flexible knife guide.

10. The surgical stapling apparatus of claim 1, wherein a proximal end portion of the channel member supports a proximal lockout assembly, and wherein the proximal lockout assembly includes a lockout bar and at least one spring supported on the lockout bar, the actuation sled configured to depress the lockout bar against the bias of the at least one spring to enable the working member to translate past the proximal lockout assembly.

11. A tool assembly for a surgical stapling apparatus, the tool assembly defining a longitudinal axis and comprising:
an anvil assembly; and
a cartridge assembly pivotably supported in relation to the anvil assembly to facilitate movement of the tool assembly between an unclamped position and a clamped position, the cartridge assembly including a staple cartridge and a channel member, the staple cartridge received within the channel member, the staple cartridge including a cartridge body, staples, an actuation sled, and a knife bar, the cartridge body defining staple receiving pockets, the staples received within the staple receiving pockets, the actuation sled movable from a sled retracted position to a sled advanced position to eject the staples from the cartridge body, the channel member including a flexible knife guide and defining a lockout groove, the flexible knife guide configured to urge a working member of a drive assembly in a transverse direction across the longitudinal axis of the tool assembly and toward the lockout groove for selectively locking out the working member.

12. The tool assembly of claim 11, wherein the knife bar includes a proximal finger that is configured to selectively engage the working member to prevent the working member from engaging the lockout groove when the knife bar is disposed in a first predetermined position.

13. The tool assembly of claim 12, wherein when the knife bar is not disposed in the first predetermined position, or when the actuation sled is not disposed in a second predetermined position, or when the knife bar is not disposed in the first predetermined position and the actuation sled is not disposed in the second predetermined position, the flexible knife guide is configured to urge the working member into the lockout groove.

14. The tool assembly of claim 13, wherein the proximal finger is configured to enable the working member to move distally beyond the lockout groove.

15. The tool assembly of claim 13, wherein when the tool assembly is disposed in the clamped position and the knife bar is not disposed the first predetermined position, the flexible knife guide is configured to urge the working member transversely relative the longitudinal axis of the tool assembly.

16. The tool assembly of claim 11, wherein the lockout groove is configured to receive a locking tooth of the working member to prevent distal movement of the working member through the tool assembly.

17. The tool assembly of claim 14, wherein the channel member defines a clearance cutout that selectively receives a distal tab of the flexible knife guide.

18. The tool assembly of claim 11, wherein the anvil assembly includes an anvil and an anvil cover, the anvil cover including a second flexible knife guide that is configured to engage the working member for selectively urging the working member in a proximal relief defined in the anvil.

19. The tool assembly of claim 11, wherein a proximal end portion of the channel member supports a proximal lockout assembly, and wherein the proximal lockout assembly includes a lockout bar and at least one spring supported on the lockout bar, the actuation sled configured to depress the lockout bar against the bias of the at least one spring to enable the working member to translate past the proximal lockout assembly.

20. A tool assembly, comprising:
an anvil assembly; and
a cartridge assembly pivotably supported in relation to the anvil assembly to facilitate movement of the tool assembly between an unclamped position and a clamped position, the cartridge assembly including a staple cartridge and a channel member, the staple cartridge received within the channel member, the staple cartridge including a cartridge body, staples, an actuation sled, and a knife bar, the actuation sled movable from a sled retracted position to a sled advanced position to eject the staples from the cartridge body, the channel member including a knife guide and defining a lockout groove, the knife guide being pivotably connected to the channel member and moveable in a transverse direction across a longitudinal axis of the tool assembly and toward the lockout groove for preventing the tool assembly from firing.
